SEQUENCE LISTING (6 sequences)

SEQ ID NO: 1 - DNA, 41 bp, Homo sapiens
  misc_feature (1)..(41)
ttcccgtcgctatcaaggaattaagagaagcaacatctccg

SEQ ID NO: 2 - DNA, 26 bp, Homo sapiens
  misc_feature (1)..(26)
ttcccgtcgctatcaagacatctccg

SEQ ID NO: 3 - DNA, 26 bp, Homo sapiens
  misc_feature (1)..(26)
ttcccgtcgctatcaagacatctccg

SEQ ID NO: 4 - DNA, 45 bp, Homo sapiens
  misc_feature (1)..(45)
tccaccgtgcagctcatcacgcagctcatgcccttcggctgcctc

SEQ ID NO: 5 - DNA, 45 bp, Homo sapiens; identical to SEQ ID NO: 4
  misc_feature (1)..(45)

SEQ ID NO: 6 - DNA, 45 bp, Homo sapiens; identical to SEQ ID NO: 4
  misc_feature (1)..(45)

(19) United States
(12) Patent Application Publication
(10) Pub. No.: US 2018/0100153 A1
(43) Pub. Date: Apr. 12, 2018

(54) Method and kit for use in inhiiting tumor progression, predicting or determining tumor progression state in VGF expressing cancers

(71) Applicants: Yu-Ting Chou, Hsinchu City (TW); Truebio LLC, College Park, MD (US)

(72) Inventors: Yu-Ting Chou, Hsinchu City (TW); Richard K. Lee, Hsinchu City (TW)

(73) Assignees: Truebio LLC, College Park, MD (US); Yu-Ting Chou, Hsinchu City (TW)

(21) Appl. No.: 15/726,711

(22) Filed: Oct. 6, 2017

Related U.S. Application Data
(60) Provisional application No. 62/405,242, filed on Oct. 7, 2016.

(51) Int. Cl. C12N 15/113; C12Q 1/6886; G01N 33/574; A61P 35/00

(52) U.S. Cl. CPC C12N 15/1136; C12Q 1/6886; G01N 33/5748; G01N 33/57423; G01N 2333/48; A61P 35/00; C12N 2310/122; C12Q 2600/158; G01N 33/57415

(57) ABSTRACT

The present invention provides a method of inhibiting tumor progression in a subject suffering from VGF expressing cancers. The present invention also provides a method and a kit of predicting or determining tumor progression state in a subject suffering from VGF expressing cancers.

### CROSS-REFERENCES TO RELATED APPLICATIONS

The present application claims priority to U.S. Provisional Appl. No. U.S. 62/405,242 filed on Oct. 7, 2016, incorporated herein by reference in its entirety. This application also contains a Sequence Listing in computer readable form. The computer readable form is incorporated herein by reference.

### BACKGROUND OF THE INVENTION

### Field of the Invention

The present invention relates to method of inhibiting tumor progression in a subject suffering from VGF expressing cancers, and method and kit of predicting or determining tumor progression state in a subject suffering from VGF expressing cancers.

### Description of Prior Art

Activating mutations in epidermal growth factor receptor (EGFR) constitute one of the major subsets among those molecular aberrations preferentially occurring in patients with clinicopathological characteristics of lung adenocarcinoma (References 1 to 4). EGFR-tyrosine kinase inhibitors (TKIs), such as gefitinib, erlotinib and afatinib, displayed profound therapeutic responses in lung adenocarcinoma harboring EGFR mutations (exon 19 deletions or the L858R mutation) (References 5 to 10). Despite this initial response, patients with EGFR mutated lung adenocarcinoma will ultimately develop resistance to EGFR-TKIs.

To date, a secondary mutation in EGFR (T790M), which abrogates the inhibitory activity of the TKIs, is reported to be the major contribution to the development of acquired resistance to EGFR-TKIs (References 11 to 13). However, the mutation of T790M infers better survival outcomes and negatively correlates with distant metastasis, thereby predicting a favorable prognosis in lung cancer patients (References 14 to 17). Thus, other non-T790M factors may affect cancer dissemination and cancer cell survival during EGFR-TKI treatment. Several studies revealed that epithelial-to-mesenchymal transition (EMT), a pro-invasive status, can endow EGFR-mutated lung cancer cells with TKI-resistance (References 18 to 19). In addition, pathological transformation from adenocarcinoma toward neuroendocrine lineage has been detected in some specimens during EGFR-TKI treatments (References 13, 20 to 22). Nonetheless, the biological underpinnings of the neuroendocrine transformation or EMT during the development of EGFR-TKI resistance were elusive.

The VGF (Nerve Growth Factor-Inducible) gene encodes a neuroendocrine protein that is secreted in normal neuroendocrine cells, responsible for energy balance and metabolism (References 23 to 24). VGF expression enhances neuronal growth and prevents apoptosis (References 25 to 26). VGF has been detected in several neuroendocrine cells and related cancers (References 27 to 29); however, the role of VGF in tumor initiation and progression is not known. Lung adenocarcinoma does not belong to neuroendocrine lineage; thus, VGF, a neuroendocrine protein, should not be expressed and detected in typical lung adenocarcinoma.

### REFERENCES

1. Hsiao S H, Lin H C, Chou Y T, Lin S E, Kuo C C, Yu M C, et al. Impact of epidermal growth factor receptor mutations on intracranial treatment response and survival after brain metastases in lung adenocarcinoma patients. Lung Cancer 2013; 81(3):455-61.
2. Pao W, Miller V, Zakowski M, Doherty J, Politi K, Sarkaria I, et al. EGF receptor gene mutations are common in lung cancers from “never smokers” and are associated with sensitivity of tumors to gefitinib and erlotinib. Proc Natl Acad Sci USA 2004; 101(36):13306-11.
3. Lynch T J, Bell D W, Sordella R, Gurubhagavatula S, Okimoto R A, Brannigan B W, et al. Activating mutations in the epidermal growth factor receptor underlying responsiveness of non-small-cell lung cancer to gefitinib. N Engl J Med 2004; 350(21):2129-39.
4. Paez J G, Janne P A, Lee J C, Tracy S, Greulich H, Gabriel S, et al. EGFR mutations in lung cancer: correlation with clinical response to gefitinib therapy. Science 2004; 304(5676):1497-500.
5. Mok T S, Wu Y L, Thongprasert S, Yang C H, Chu D T, Saijo N, et al. Gefitinib or carboplatin-paclitaxel in pulmonary adenocarcinoma. N Engl J Med 2009; 361(10):947-57.
6. Maemondo M, Inoue A, Kobayashi K, Sugawara S, Oizumi S, Isobe H, et al. Gefitinib or chemotherapy for non-small-cell lung cancer with mutated EGFR. N Engl J Med 2010; 362(25):2380-8.
7. Zhou C, Wu Y L, Chen G, Feng J, Liu X Q, Wang C, et al. Erlotinib versus chemotherapy as first-line treatment for patients with advanced EGFR mutation-positive non-small-cell lung cancer (OPTIMAL, CTONG-0802): a multicentre, open-label, randomised, phase 3 study. Lancet Oncol 2011; 12(8):735-42.
8. Sequist L V, Yang J C, Yamamoto N, O'Byrne K, Hirsh V, Mok T, et al. Phase III study of afatinib or cisplatin plus pemetrexed in patients with metastatic lung adenocarcinoma with EGFR mutations. J Clin Oncol 2013; 31(27):3327-34.
9. Wu Y L, Zhou C, Hu C P, Feng J, Lu S, Huang Y, et al. Afatinib versus cisplatin plus gemcitabine for first-line treatment of Asian patients with advanced non-small-cell lung cancer harbouring EGFR mutations (LUX-Lung 6): an open-label, randomised phase 3 trial. Lancet Oncol 2014; 15(2):213-22.
10. Rosell R, Carcereny E, Gervais R, Vergnenegre A, Massuti B, Felip E, et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol 2012; 13(3):239-46.
11. Arcila M E, Oxnard G R, Nafa K, Riely G J, Solomon S B, Zakowski M F, et al. Rebiopsy of lung cancer patients with acquired resistance to EGFR inhibitors and enhanced detection of the T790M mutation using a locked nucleic acid-based assay. Clin Cancer Res 2011; 17(5):1169-80.
12. Kobayashi S, Boggon T J, Dayaram T, Janne P A, Kocher O, Meyerson M, et al. EGFR mutation and resistance of non-small-cell lung cancer to gefitinib. N Engl J Med 2005; 352(8):786-92.
13. Sequist L V, Waltman B A, Dias-Santagata D, Digumarthy S, Turke A B, Fidias P, et al. Genotypic and histological evolution of lung cancers acquiring resistance to EGFR inhibitors. Sci Transl Med 2011; 3(75):75ra26.
14. Antonicelli A, Cafarotti S, Indini A, Galli A, Russo A, Cesario A, et al. EGFR-targeted therapy for non-small cell lung cancer: focus on EGFR oncogenic mutation. Int J Med Sci 2013; 10(3):320-30.
15. Uramoto H, Yano S, Tanaka F. T790M is associated with a favorable prognosis in Japanese patients treated with an EGFR-TKI. Lung Cancer 2012; 76(1):129-30.
16. Kuiper J L, Heideman D A, Thunnissen E, Paul M A, van Wijk A W, Postmus P E, et al. Incidence of T790M mutation in (sequential) rebiopsies in EGFR-mutated NSCLC-patients. Lung Cancer 2014; 85(1):19-24.
17. Li W, Ren S, Li J, Li A, Fan L, Li X, et al. T790M mutation is associated with better efficacy of treatment beyond progression with EGFR-TKI in advanced NSCLC patients. Lung Cancer 2014; 84(3):295-300.
18. Yauch R L, Januario T, Eberhard D A, Cavet G, Zhu W, Fu L, et al. Epithelial versus mesenchymal phenotype determines in vitro sensitivity and predicts clinical activity of erlotinib in lung cancer patients. Clin Cancer Res 2005; 11(24 Pt 1):8686-98.
19. Thomson S, Buck E, Petti F, Griffin G, Brown E, Ramnarine N, et al. Epithelial to mesenchymal transition is a determinant of sensitivity of non-small-cell lung carcinoma cell lines and xenografts to epidermal growth factor receptor inhibition. Cancer Res 2005; 65(20):9455-62.
20. Shiao T H, Chang Y L, Yu C J, Chang Y C, Hsu Y C, Chang S H, et al. Epidermal growth factor receptor mutations in small cell lung cancer: a brief report. J Thorac Oncol 2011; 6(1):195-8.
21. Tatematsu A, Shimizu J, Murakami Y, Horio Y, Nakamura S, Hida T, et al. Epidermal growth factor receptor mutations in small cell lung cancer. Clin Cancer Res 2008; 14(19):6092-6.
22. Kogo M, Shimizu R, Uehara K, Takahashi Y, Kokubo M, Imai Y, et al. Transformation to large cell neuroendocrine carcinoma as acquired resistance mechanism of EGFR tyrosine kinase inhibitor. Lung Cancer 2015; 90(2):364-8.
23. Hahm S, Mizuno T M, Wu T J, Wisor J P, Priest C A, Kozak C A, et al. Targeted deletion of the Vgf gene indicates that the encoded secretory peptide precursor plays a novel role in the regulation of energy balance. Neuron 1999; 23(3):537-48.
24. Bartolomucci A, La Corte G, Possenti R, Locatelli V, Rigamonti A E, Torsello A, et al. TLQP-21, a VGF-derived peptide, increases energy expenditure and prevents the early phase of diet-induced obesity. Proc Natl Acad Sci USA 2006; 103(39):14584-9.
25. Severini C, Ciotti M T, Biondini L, Quaresima S, Rinaldi A M, Levi A, et al. TLQP-21, a neuroendocrine VGF-derived peptide, prevents cerebellar granule cells death induced by serum and potassium deprivation. J Neurochem 2008; 104(2):534-44.
26. Shimazawa M, Tanaka H, Ito Y, Morimoto N, Tsuruma K, Kadokura M, et al. An inducer of VGF protects cells against E R stress-induced cell death and prolongs survival in the mutant SOD1 animal models of familial ALS. PLoS One 2010; 5(12):e15307.
27. Rindi G, Licini L, Necchi V, Bottarelli L, Campanini N, Azzoni C, et al. Peptide products of the neurotrophin-inducible gene vgf are produced in human neuroendocrine cells from early development and increase in hyperplasia and neoplasia. J Clin Endocrinol Metab 2007; 92(7):2811-5.
28. Matsumoto T, Kawashima Y, Nagashio R, Kageyama T, Kodera Y, Jiang S X, et al. A new possible lung cancer marker: VGF detection from the conditioned medium of pulmonary large cell neuroendocrine carcinoma-derived cells using secretome analysis. Int J Biol Markers 2009; 24(4):282-5.
29. Annaratone L, Medico E, Rangel N, Castellano I, Marchio C, Sapino A, et al. Search for neuro-endocrine markers (chromogranin A, synaptophysin and VGF) in breast cancers. An integrated approach using immunohistochemistry and gene expression profiling. Endocr Pathol 2014; 25(3):219-28.

### SUMMARY OF THE INVENTION

The present invention provides a method of inhibiting tumor progression in a subject suffering from VGF expressing cancers, comprising administering an antagonist of VGF to the subject.

The present invention also provides a method predicting or determining tumor progression state in a subject suffering from cancer, comprising: (a) providing a sample from the subject; and (b) measuring an expression level of VGF gene in the sample from the subject using reagents specific for VGF gene product that are selected from the group consisting of probes, primers, antibodies, antibody fragments and antibody coated beads, wherein the VGF gene product is VGF mRNA or VGF protein expression, wherein positive detection of VGF gene product is indicative of tumor progression.

The present invention further provides a kit for predicting or determining tumor progression state in a subject suffering from cancer comprising reagent specific for VGF gene product, wherein the reagent specific for VGF gene product comprises an antibody against VGF protein, a nucleic acid probe for hybridizing to VGF mRNA, a primer pair for amplifying VGF cDNA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates development of EGFR-TKI resistance and Epithelial-mesenchymal transdifferentiation in lung cancer cells. (FIG. 1A) IC₅₀ analysis of gefitinib, erlotinib, afatinib, AZD9291 or rociletinib in HCC827 and HCC827GR cells via alamarBlue® assay. (FIG. 1B) Clonogenic analysis of HCC827 and HCC827GR cells treated with indicated concentrations of gefitinib, erlotinib, afatinib, or AZD9291 for 10 days. Photographs represent growth of HCC827 and HCC827GR cells stained by crystal violet. (FIG. 1C) Immunoblotting analysis (upper) for assessing the expression of phosphorylated EGFR (p-EGFR), total EGFR (EGFR), phosphorylated ERK1/2 (p-ERK1/2), total ERK1/2 (ERK), phosphorylated AKT (p-AKT) and total AKT (AKT) in HCC827 and HCC827GR cells treated with or without gefitinib (1 μM) for 1 hr. Immunoblotting analysis (lower) for assessing the expression of cleaved PARP and active Caspase 3, two apoptotic markers, in HCC827 and HCC827GR cells treated with or without gefitinib (1 μM) for 24 hr. (FIG. 1D) Representative phase-contrast images of HCC827 and HCC827GR cells. Scale bar, 100 μm. (FIG. 1E) Immunofluorescence analysis for assessing the expression of E-cadherin (E-cad, green) and Vimentin (VIM, red) expressions in HCC827GR versus HCC827 cells. Nuclei were stained in blue with DAPI. Scale bar, 100 μm. (FIG. 1F) Q-PCR analysis for measuring mRNA levels of CDH1 (E-cad), EPCAM (EpCAM), Vimentin (VIM) and TWIST1 in HCC827GR versus HCC827 cells.

FIG. 2 illustrates decreased barrier function and enhanced cancer dissemination in EGFR-TKI resistant cells. (FIG. 2A) ECIS analysis for measuring impedance (upper left) and monitoring the change of Rb (barrier function; upper right) in HCC827GR versus HCC827 cells. The representative values of Rb and Alpha (Cell-extracellular matrix interaction) were listed (bottom). (FIG. 2B) Cell tracking analysis for measuring the relative migratory distance of HCC827 versus HCC827GR cells during 24 hr. Asterisks indicate statistical significance: **p<0.01. (FIG. 2C) Wound-healing assay of HCC827 and HCC827GR cells. Asterisks indicate statistical significance: *p<0.05. (FIG. 2D) Trans-well migration assay of HCC827 and HCC827GR cells. Asterisks indicate statistical significance: ***p<0.001. (FIG. 2E) Trans-well invasion analysis of HCC827 and HCC827GR cells. Asterisks indicate statistical significance: ***p<0.001.

FIG. 3 illustrates expression of VGF in EGFR-TKI resistant lung cancer cells. (FIG. 3A) Quantitative real-time PCR (Q-PCR) analysis (left), and immunoblotting (right) for measuring the expression of VGF in HCC827GR versus and HCC827 cells. Tubulin served as a loading control. (FIG. 3B) Q-PCR analysis (left) for measuring the expression of VGF in HCC827GR-2, an independently selected EGFR-TKI resistant HCC827 pool, versus and HCC827 cells. Tubulin served as a loading control. Gene expression analysis (right) for VGF expression in HCC827 and EGFR-TKI resistant clones (ER3 and T15-2) from the database of GSE38310. (FIG. 3C) Gene expression analysis for VGF expression in different subtypes of lung cancer cell lines from the database of TCGA (CCLE). SCLC: small cell lung cancer; ADC: adenocarcinoma; SCC: squamous cell carcinoma. (FIG. 3D) List of IC50 of gefitinib and EGFR mutations status (left) and Q-PCR analysis (right) for assessing VGF expression in the indicated lung adenocarcinoma cell lines. (FIG. 3E) Q-PCR analysis (left) and western blotting (right) for measuring VGF expression in HCC827GR cells infected with lentiviral vectors encoding shVGF (shVGF) or scrambled control (SC). shVGF#1 and shVGF#2 target different regions in VGF mRNA. (FIG. 3F) AlamarBlue® assay for measuring viability of HCC827 and HCC827GR cells infected with lentiviral vectors encoding shVGF (shVGF) or scrambled control (SC), followed by treatment with different concentrations of gefitinib for 3 days.

FIG. 4 illustrates that VGF encourages EGFR-TKI resistance. (FIG. 4A) Q-PCR analysis (left) and immunoblotting (right) for VGF expression in HCC827 cells infected with the lentiviral vector encoding cDNA of VGF (HCC827-VGF) or empty control vector (HCC827-Ctrl). GAPDH served as a loading control. (FIG. 4B) IC50 analysis of gefitinib, erlotinib, afatinib, AZD9291 or rociletinib in HCC827-Ctrl and HCC827-VGF cells via alamarBlue® assay. (FIG. 4C) Clonogenic analysis of HCC827-Ctrl and HCC827-VGF cells treated with indicated concentrations of gefitinib, erlotinib, afatinib, or AZD9291 for 10 days. Photographs represent growth of HCC827-Ctrl and HCC827-VGF cells stained by crystal violet. (FIG. 4D) Immunoblotting analysis (upper) for assessing the expression of phosphorylated EGFR (p-EGFR), total EGFR (EGFR), phosphorylated ERK1/2 (p-ERK1/2), total ERK1/2 (ERK) phosphorylated AKT (p-AKT) and total AKT (AKT) in HCC827-Ctrl and HCC827-VGF cells treated with or without gefitinib (1 μM) for 1 hr Immunoblotting analysis (lower) for assessing the expression of apoptotic markers, cleaved PARP and active Caspase 3, in HCC827-Ctrl and HCC827-VGF cells treated with or without gefitinib (1 μM) for 24 hr.

FIG. 5 illustrates that VGF induces EMT and cancer cell dissemination. (FIG. 5A) Representative phase-contrast images of HCC828 cells infected with the lentiviral vector encoding cDNA of VGF (HCC827-VGF) or empty control vector (HCC827-Ctrl). Scale bar, 100 μm. (FIG. 5B) Q-PCR analysis for E-cadherin (E-cad), EpCAM, and Vimentin (VIM) expression in HCC827-Ctrl and HCC827-VGF cells (left) Immunoblotting for E-cadherin (E-cad), EpCAM, Vimentin (VIM) and Twist expression in HCC827-Ctrl and HCC827-VGF cells (right). (FIG. 5C) Immunoblotting analysis in parental HCC827 (P), HCC827GR (GR), HCC827-Ctrl (Ctrl) and HCC827-VGF (VGF) cells for assessing the expression of E-cadherin (E-cad), EpCAM, Vimentin (VIM), and TWIST1. (FIG. 5D) Immunofluorescence for E-cadherin (E-cad; green) and Vimentin (VIM; red)) expression in HCC827-Ctrl (Ctrl) and HCC827-VGF (VGF) cells. Nuclei were stained in blue with DAPI. Scale bar, 100 μm. (FIG. 5E) ECIS analysis in HCC827-VGF versus HCC827-Ctrl cells for monitoring the change of impedance (upper left) and Rb (barrier function; upper right). The representative values of Rb and Alpha (Cell-extracellular matrix interaction) were listed (bottom). (FIG. 5F) Trans-well migration assay of HCC827 and HCC827GR cells. Asterisks indicate statistical significance: ***p<0.001. (FIG. 5G) Trans-well matrigel invasion analysis of HCC827 and HCC827GR cells. Asterisks indicate statistical significance: ***p<0.001.

FIG. 6 illustrates that VGF-silencing attenuates tumor cell growth in vitro and in vivo. (FIG. 6A) Clonogenic assay for assessing the effect of VGF-silencing on EGFR-TKI resistant HCC827GR (upper) and H1975 (lower) lung cancer cells. HCC827GR and H1975 cells were infected with lentiviral vector encoding shVGF (shVGF) or scrambled control (SC) and subjected to clonogenic analysis. shVGF#1 and shVGF#2 target different regions in VGF mRNA Photographs represent growth of cells stained by crystal violet. (FIG. 6B) Xenograft assay for assessing the effect of VGF-silencing on tumor growth. HCC827GR cells were fist ted with lentiviral vector encoding shVGF (shVGF) or scrambled control (SC) and subjected to trypan blue viability assay. Survived cells were further injected subcutaneously into nude mice. Tumor volume was monitored over time as indicated (left upper). The representative photographs illustrate xenografted tumors (white arrows) 64 days after injection (left lower). Error bars indicate the SEM (n=10 mice/group; ***P<0.001). Tumor weight was measured after harvest (right).

FIG. 7 illustrates that VGF expression correlates tumor malignancy in lung adenocarcinoma. (FIG. 7A) Representative immunohistochemistry staining (left) for weak and strong VGF expression in lung adenocarcinoma. Scale bar, 200 μm. Chi-square analysis (right) for correlation between VGF expression and tumor grades in lung adenocarcinoma. (FIG. 7B) A scatter plot generated from primary lung adenocarcinoma (GSE31548) displaying positive correlations between VGF and EMT markers, TWIST1, Vimentin (VIM), and CDH2 (Spearman correlation analysis). (FIG. 7C) Kaplan-Meier analysis for the correlation of VGF (upper) or CEACAM6 (lower) with the overall survival of primary lung adenocarcinoma from the TCGA (LUAD) cohort (log-rank analysis). (FIG. 7D) Kaplan-Meier analysis for the correlation of VGF (upper left), CEACAM6 (lower left), Synapphysin (SYP, upper right), and Chromogranin (CHGA, lower right) with the overall survival in patients of EGFR-mutated primary lung adenocarcinoma from the TCGA (LUAD) cohort (log-rank analysis). (FIG. 7E) mRNA in situ hybridization analysis for VGF mRNA expression in EGFR-TKI resistant lung adenocarcinomas, harboring EGFR mutations.

FIG. 8 illustrates lack of T790M and amplification of MET and HER2 in HCC827GR cells. (FIG. 8A) Direct DNA sequencing analysis of EGFR exon 19 and exon 20 from HCC827 and HCC827GR cells. The comparison of EGFR exon 19 and exon 20 from HCC827 and HCC827GR cells with those from reference sequences displayed that both HCC827 and HCC827GR contained EGFR deletion (delE746_A750) in exon 19 (upper), while both of them lacked T790M mutation in exon 20 (middle and lower). (FIG. 8B) Q-PCR analysis for assessing the relative DNA copy numbers of MET (left), EGFR (middle), and HER2 (right) in HCC82GR versus HCC827 cells.

FIG. 9 illustrates rociletinib-resistance in HCC827GR compared to HCC827 cells. (FIG. 9A) Clonogenic analysis of HCC827 and HCC827GR cells treated with indicated concentrations of rociletinib for 10 days. Photographs represent growth of HCC827-VGF and HCC827-Ctrl cells stained by crystal violet. (FIG. 9B) Clonogenic analysis of rociletinib for 10 days. Photographs represent growth of HCC827-Ctrl and HCC827-VGF cells stained by crystal violet.

FIG. 10 illustrates detecting expression of VGF expression in EGFR-TKI resistant cells and adenocarcinoma mixed with neuroendocrine cells by mRNA in situ hybridization (mISH). (FIG. 10A) mISH analysis for VGF mRNA expression in HCC827 (EGFR-TKI sensitive), HCC827GR (resistant) and H1975 (resistant) cells, showing that VGF mRNA was expressed in HCC827GR and H1975, but not in HCC827 cells. (FIG. 10B) mISH analysis for VGF mRNA expression in a lung adenocarcinoma mixed with neuroendocrine cells.

FIG. 11 illustrates EGFR-TKI resistance in HCC827GR-2, an independent pool. (FIG. 11A) Q-PCR analysis for VGF expression in HCC827GR-2 cells. HCC827GR-2 cells were independently obtained from HCC827 under gefitinib (500 nM) selection for 3 weeks. (FIG. 11B) Clonogenic analysis of HCC827GR-2 versus HCC827 cells treated with indicated concentrations of gefitinib, erlotinib, or afatinib for 10 days. Photographs represent growth of HCC827-Ctrl and HCC827-VGF cells stained by crystal violet.

FIG. 12 illustrates effect of VGF expression on cell survival. (FIG. 12A) Imunomagnetic reduction (IMR) analysis for assessing the expression of secreted VGF in conditioned media from HCC827 and HCC827GR cells. (FIG. 12B) Clonogenic analysis of HCC827GR cells infected with lentiviral vector encoding scrambled control (left) or shVGF (right). Cells were further subjected to clonogenic assay under the growth of supplement with condition media (CM) from HEK293T cells transfected with expression vector encoding VGF cDNA (VGF) or empty control (Ctrl) vector for 14 days. Colonies were analyzed and quantified by Imaging J software. Asterisks indicate statistical significance: *p<0.05. (FIG. 12C) Condition media (CM) were collected from HCC827, HCC827GR, HCC827-Ctrl and HCC827-VGF cells under the growth of RPMI supplemented with 1% FBS. HCC827 cells were subjected to clonogenic assay under the growth of CM from HCC827, HCC827GR, HCC827-Ctrl or HCC827-VGF cells for 14 days. Colonies were analyzed and quantified by Imaging J software. Asterisks indicate statistical significance: *p<0.05.

FIG. 13 illustrates VGF as a therapeutic target. (FIG. 13A) Q-PCR (left) analysis for VGF expression and clonogenic assay (right) in HCC827GR/tet-on control cells. HCC827GR cells were stably transfected with pLKO-tet-on control vector, to generate HCC827GR/tet-on control cells in which endogenous VGF levels were not downregulated by treatment with doxycycline. (FIG. 13B) Xenograft assay for assessing the effect of doxycycline treatment on tumor growth. HCC827GR/tet-on control cells were injected subcutaneously into nude mice. 32 days after cancer cell injection, mice were treated with or without daily oral doxycycline (Dox) for another 30 days. Tumor volume was monitored over time as indicated (left). Tumor weight was measured after harvest (upper right). The representative photographs illustrate tumor growth 30 days after Dox or normal saline treatment (lower right). ns means no significant (n=6 mice/group). (FIG. 13C) Q-PCR (left) analysis for VGF expression and clonogenic assay (right) in HCC827GR/tet-on shVGF cells in which shVGF was induced by doxycycline (Dox). HCC827GR cells were stably transfected with pLKO-tet-on-shVGF, which encodes a doxycycline (Dox)-inducible shVGF, to generate HCC827GR/tet-on-shVGF cells in which endogenous VGF levels could be downregulated by treatment with doxycycline (FIG. 13D) Xenograft assay for assessing the effect of VGF-silencing on tumor growth. HCC827GR/tet-on-shVGF cells were injected subcutaneously into nude mice. 32 days after cancer cell injection, mice were treated with or without daily oral doxycycline (Dox) for another 30 days. Tumor volume was monitored over time as indicated (left). Tumor weight was measured after harvest (upper right). The representative photographs illustrate tumor growth 30 days after Dox or normal saline treatment (lower right). Scale bar, 1 mm. Error bars indicate the SEM (n=6 mice/group; *P<0.05).

FIG. 14 illustrates that VGF positively and negatively correlated with EMT markers and CEACAM6, respectively, in lung adenocarcinoma. (FIG. 14A), (FIG. 14B) A scatter plot generated from primary lung adenocarcinoma displaying positive correlations between VGF, TWIST1 (FIG. 14A upper and lower), VIM (FIG. 14B, upper) and CDH2 levels (FIG. 14B, lower) (Spearman correlation analysis). (FIG. 14C) Q-PCR analysis for CEACAM6 expression in HCC827GR versus HCC827 cells (right). Gene expression analysis (right) for CEACAM6 expression in HCC827 and EGFR-TKI resistant clones (ER3 and T15-2) from the database of GSE38310. (FIG. 14D) A scatter plot generated from primary lung adenocarcinoma displaying positive correlations between VGF and CEACAM6 levels (Spearman correlation analysis).

FIG. 15 illustrates lack of correlation of SYP and CHGA with survival in lung adenocarcinoma. Kaplan-Meier analysis for the correlation of SYP (left) and CHGA (right) with the overall survival of primary lung adenocarcinoma from the TCGA (LUAD) cohort (log-rank analysis).

FIG. 16 illustrates that VGF induces TWIST1 to encourage EGFR-TKI resistance. (FIG. 16A) Q-PCR analysis for TWIST1, SNAIL, and SLUG expression in HCC827GR versus HCC827 cells. (FIG. 16B) A scatter plot generated from primary lung adenocarcinoma (HCC827) displaying positive correlations between VGF and TWIST1, Vimentin (VIM), and CDH2 (Spearman correlation analysis). (FIG. 16C) Q-PCR analysis for E-cad, EpCAM, VIM, and TWIST1 expression in HCC827 cells infected with the lentiviral vector encoding cDNA of VGF (HCC827-VGF) or empty control vector (HCC827-Ctrl). (FIG. 16D) Clonogenic analysis of HCC827 cells infected with the lentiviral vector encoding cDNA of TWIST1 (TWIST1) or empty control vector (Ctrl), treated with indicated concentrations of gefitinib, erlotinib, or afatinib for 10 days.

FIG. 17 illustrates VGF expression in breast cancer and lung cancer. (FIG. 17A) Kaplan-Meier analysis for the correlation of VGF with the overall survival of breast cancer. (FIG. 17B) Q-PCR analysis for VGF expression in breast cancer cells (MCF-7, MB-453, MB-231), and lung cancer cells (HCC827, HCC827GR). (FIG. 17C) Q-PCR (left) analysis for VGF expression and clonogenic assay (right) in MCF-7 cells infected with lentiviral vectors encoding shVGF (shVGF) or scrambled control (SC). shVGF#1 and shVGF#2 target different regions in VGF mRNA.

FIG. 18 illustrates effect of VGF mutants on low serum stress. (FIG. 18A) Schematic representation of VGF deletion mutations (FIG. 18B) A table summarizing clonogenic analysis of HEK293T cells transfected with expression vector encoding empty control (Ctrl), full-length VGF cDNA (VGF), or truncated VGF cDNA as described in the FIG. 18A under the growth of DMEM supplement with 1% FBS for 10 days.

### DETAILED DESCRIPTION OF THE INVENTION

In the present invention, the inventors discovered that VGF was highly expressed in VGF expressing cancers such as EGFR-TKI resistant lung adenocarcinoma cells and associated with EMT. The role of VGF in tumor progression in VGF expressing cancers were further characterized.

The present invention provides a method of inhibiting tumor progression in a subject suffering from VGF expressing cancers, comprising administering an antagonist of VGF to the subject.

In a preferred embodiment, the antagonist of VGF is antibody, small molecule compound, siRNA, shRNA, or antisense RNA against VGF.

In another preferred embodiment, the tumor progression comprises tumor growth, cancer dissemination, metastasis and drug resistance.

In another preferred embodiment, the drug resistance comprises EGFR-TKI resistance.

In another preferred embodiment, the VGF-expressing cancers comprise VGF-expressing cancers originated from lung, breast, or other different organs.

The present invention also provides a method of predicting or determining tumor progression state in a subject suffering from VGF expressing cancer, comprising: (a) providing a sample from the subject; and (b) measuring an expression level of VGF gene in the sample from the subject using reagents specific for VGF gene product that are selected from the group consisting of probes, primers, antibodies, antibody fragments and antibody coated beads, wherein the VGF gene product is VGF mRNA or VGF protein expression, wherein positive detection of VGF gene product is indicative of tumor progression.

In a preferred embodiment, the expression level of VGF gene is determined by quantitative real-time PCR or in situ hybridization for VGF mRNA.

In another preferred embodiment, the expression level of VGF gene is determined by immunoblotting, immunohistochemistry, or immunomagnetic reduction for VGF protein.

In another preferred embodiment, the sample comprises tissue sample, serum, pleural effusion, ascites, or other body fluids.

The present invention further provides a kit for predicting or determining tumor progression state in a subject suffering from VGF expressing cancers comprising reagent specific for VGF gene product, wherein the reagent specific for VGF gene product comprises an antibody against VGF protein, a nucleic acid probe for hybridizing to VGF mRNA, a primer pair for amplifying VGF cDNA.

In another preferred embodiment, the VGF-expressing cancers comprise VGF-expressing cancers originated from lung, breast, or other different organs.

The present invention also provides a pharmaceutical composition for inhibiting tumor progression in a subject suffering from VGF expressing cancers, comprising an antagonist of VGF to the subject. In a preferred embodiment, the antagonist of VGF is antibody, small molecule compound, siRNA, shRNA, or antisense RNA against VGF.

The “tumor progression” herein is refer to the third and last phase in tumor development. This phase is characterized by increased growth speed and invasiveness of the tumor cells, including tumor growth, cancer dissemination, and drug resistance, such as EGFR-TKI resistance.

The present invention provides a method of reducing resistance for EGFR tyrosine kinase inhibitor-resistant cancer in a subject which has a tumor expressing mutated forms of the EGFR and has acquired resistance to tyrosine kinase inhibitor (TKI) treatment, comprising administering a pharmaceutical composition comprising an antibody against VGF.

In a preferred embodiment, the EGFR tyrosine kinase inhibitor-resistant cancer is lung cancer.

In another preferred embodiment, the lung cancer is adenocarcinoma.

The present invention also provides a pharmaceutical composition for reducing resistance for EGFR tyrosine kinase inhibitor-resistant cancer in a subject which has a tumor expressing mutated forms of the EGFR and has acquired resistance to tyrosine kinase inhibitor (TKI) treatment, comprising an antibody against VGF.

In a preferred embodiment, the EGFR tyrosine kinase inhibitor-resistant cancer is lung cancer.

In another preferred embodiment, the lung cancer is adenocarcinoma.

In a preferred embodiment,

### Examples

The examples below are non-limiting and are merely representative of various aspects and features of the present invention.

The Involvement of Epithelial-to-Mesenchymal Transition in EGFR-TKI Resistance.

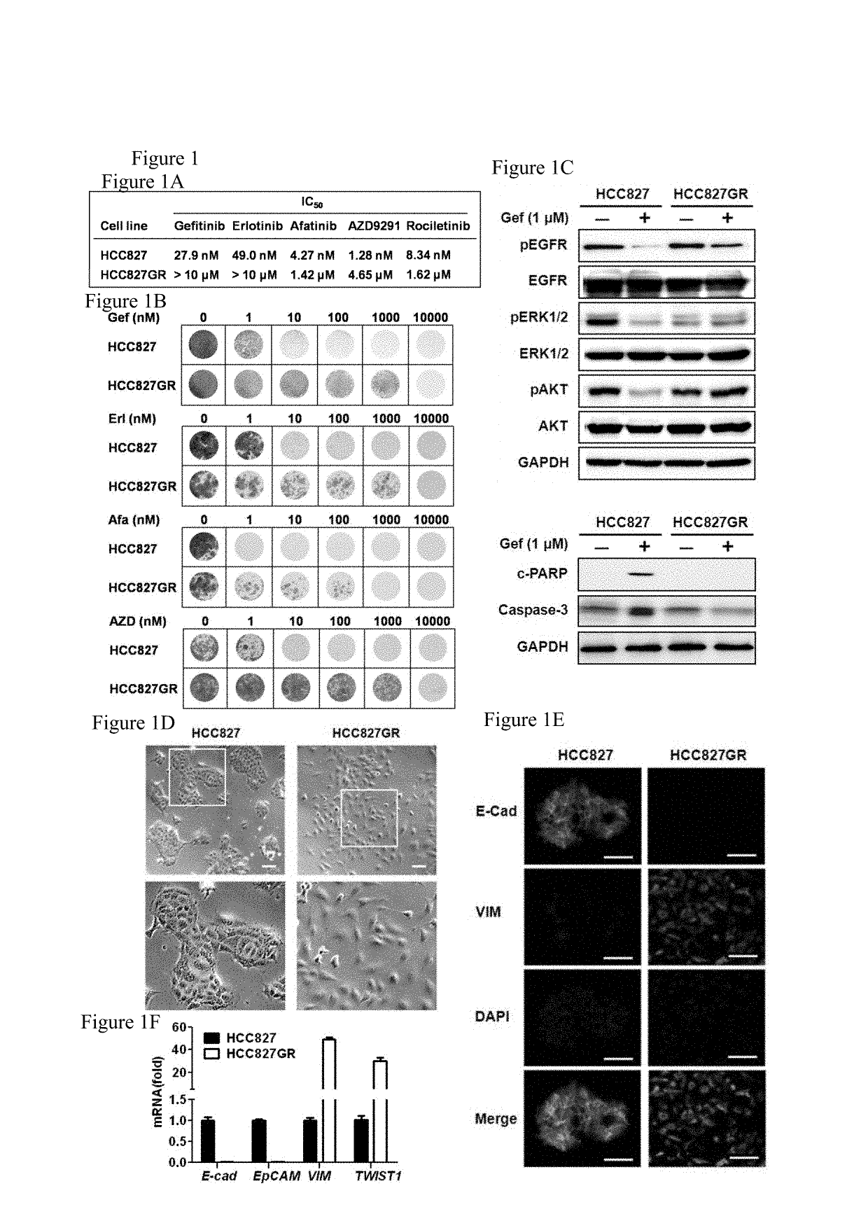

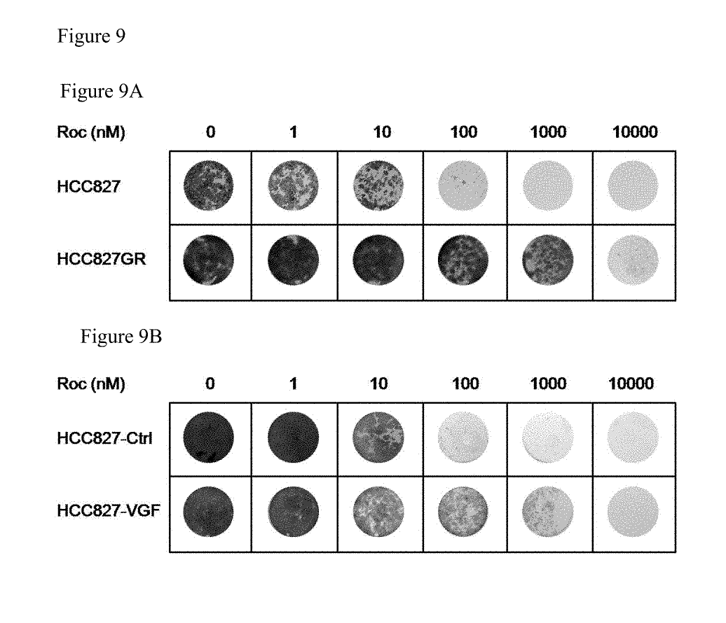

To investigate the mechanism of resistance to EGFR-TKIs in lung cancer, lung adenocarcinoma HCC827 cells, which carry EGFR delE746_A750 mutant, were treated with the stepwise increased concentration of gefitinib, and survived cells were pooled together, propagated and named as HCC827GR cells. IC50 analysis from alamarBlue® assay showed that HCC827GR cells were resistant to not only gefitinib but also erlotinib, and afatinib (FIG. 1A). Moreover, HCC827GR exhibited resistance to AZD9291 and rociletinib, the third generation of TKIs (FIGS. 1A and 9). Consistently, clonogenic assay demonstrated that HCC827GR cells survived better under the treatment of above-mentioned EGFR-TKI compared to HCC827 cells, supporting that HCC82GR cells are resistant to EGFR-TKIs (FIG. 1B). As activating phosphorylation of AKT and ERK, downstream molecules of EGFR signaling, are responsible for cellular survival and proliferation, respectively, the inventors examined the phosphorylation of EGFR, AKT and ERK in HCC827GR versus HCC827 cells Immunoblotting assay showed that upon gefitinib treatment, phosphorylation of EGFR and ERK were attenuated in HCC827 as well as in HCC827GR cells, whereas phosphorylation of AKT was not diminished by gefitinib in HCC827GR compared to HCC827 cells, suggesting the involvement of AKT signaling in EGFR-TKI resistance (FIG. 1C, upper). Western blot analysis revealed that gefitinib treatment induced the expression of activated caspase 3 and PARP, two apoptosis markers, in HCC827 but not in HC827GR cells, supporting that HCC827GR cells are resistant to gefitinib-induced apoptosis (FIG. 1C, lower).

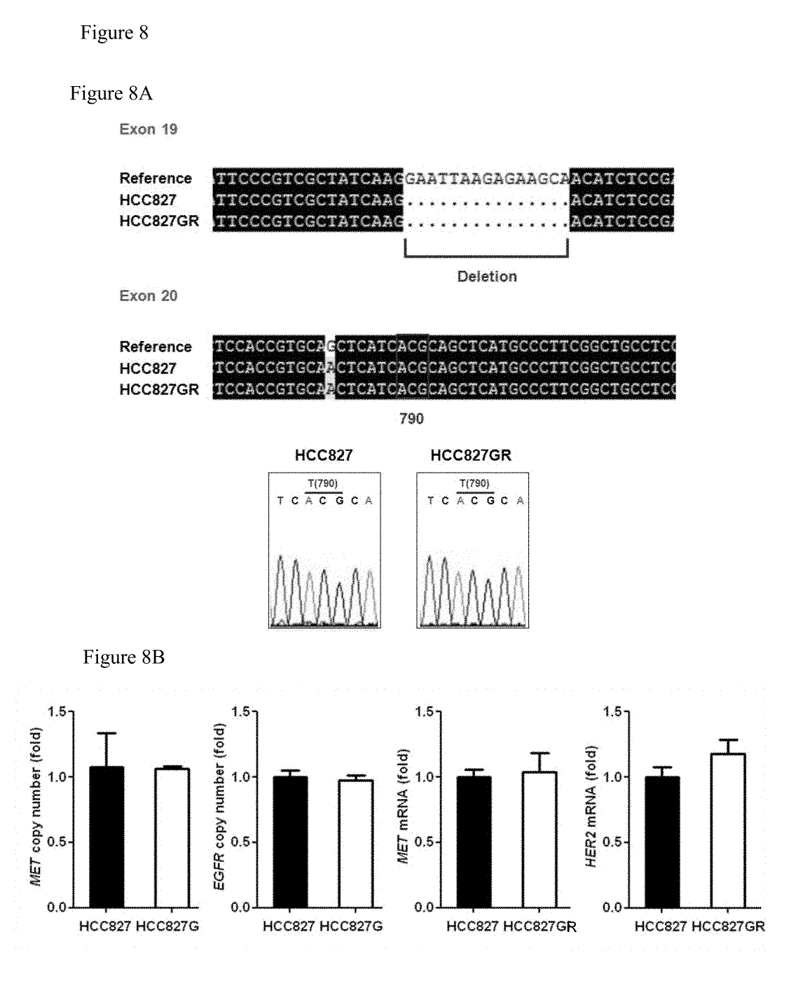

HCC827GR cells, though resistant to EGFR-TKIs, neither acquired the mutation of EGFR T790M nor amplification of MET or HER2 (FIG. 8). Phase-contrast imaging showed that HCC827GR cells contained a spindle-like phenotype, which was much different from that of the epithelial morphology in HCC827 (FIG. 1D). Q-PCR assay revealed that E-cadherin and EpCAM, two epithelial markers, were highly expressed in HCC827 but not in HCC827GR while HCC827GR contained higher levels of Vimentin and TWIST1, two mesenchymal markers, compared to HCC827 cells (FIG. 1F).

Immunofluorescence staining confirmed the reverse expression of E-cadherin and Vimentin between HCC827 and HCC827GR cells (FIG. 1E). These data indicate a possible correlation between EMT and EGFR-TKI resistance.

Loss of Barrier Function and Gain of Invasion Ability in EGFR-TKI Resistant Cells.

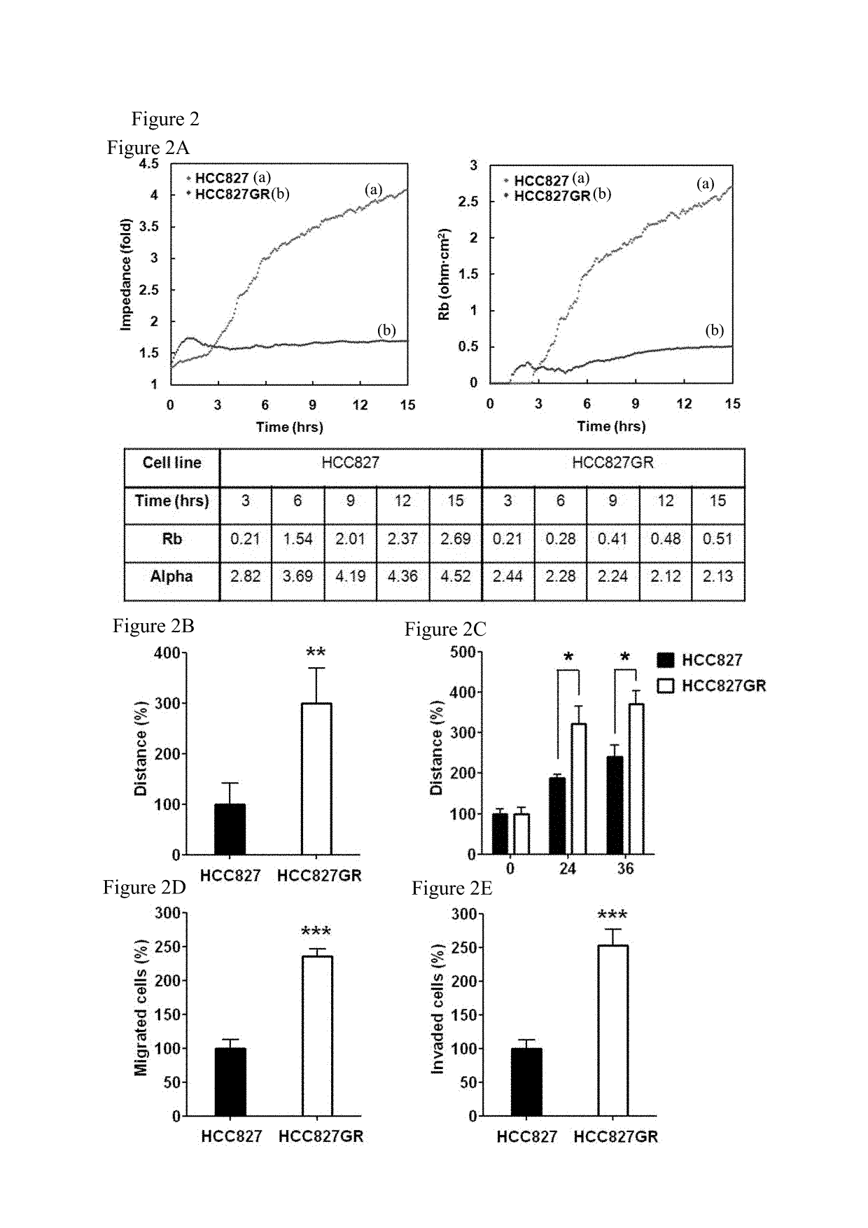

Loss of barrier function is the key cellular event of EMT. ECIS analysis revealed that after seeding, levels of impedance surged in HCC827 but not in HCC827GR cells (FIG. 2A, upper left). Impedance level is affected by the barrier function (Rb) and the passage beneath the cells (alpha). The inventors observed that huge elevation of Rb level occurred in HCC827 but not in HCC827GR cells, indicating a loss of barrier function in HCC827 GR cells (FIG. 2A, upper right and bottom). Because loss of barrier function contributes to cancer cell migration and invasion, the inventors performed migration and invasion assays in HCC827 and HCC827GR cells. Cell tracking analysis displayed that HCC827GR cells had better migration and wound healing abilities than did HCC827 cells (FIGS. 2B and C). Moreover, transwell migration and invasion assays revealed that HCC827GR cells were more migratory and invasive than HCC827 (FIGS. 2D and E). Our findings indicate that EMT-mediated EGFR-TKI resistance could contribute to migration and invasion in lung cancer cells.

VGF Expression in EGFR-TKI Resistant Lung Cancer Cells.

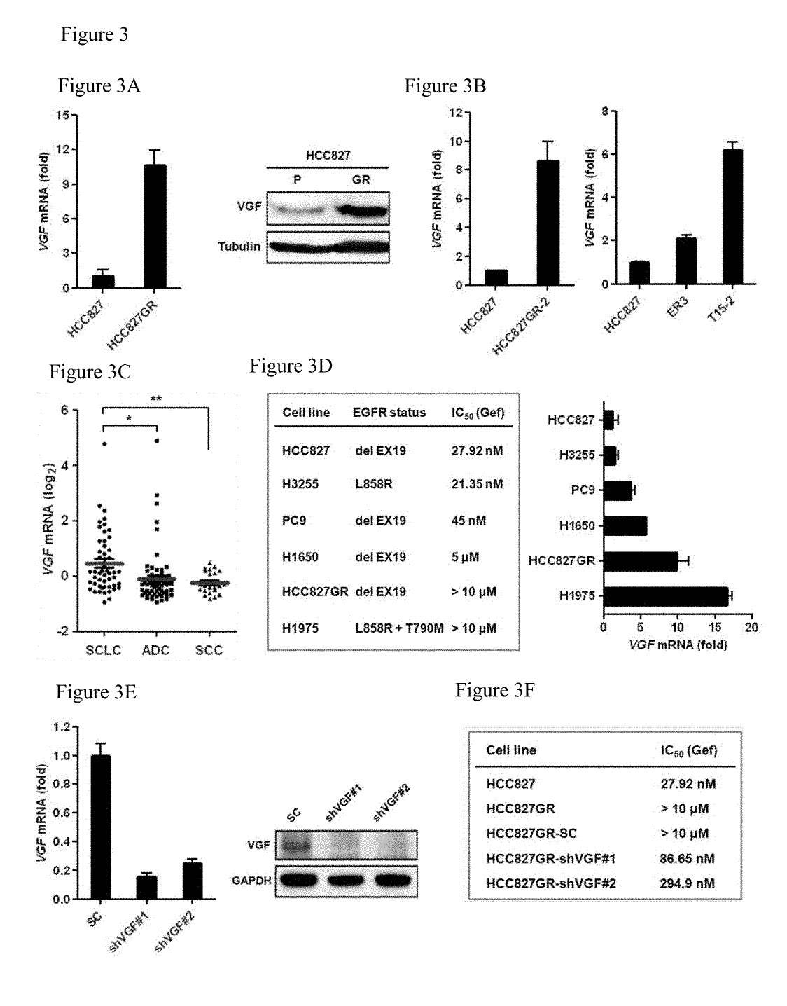

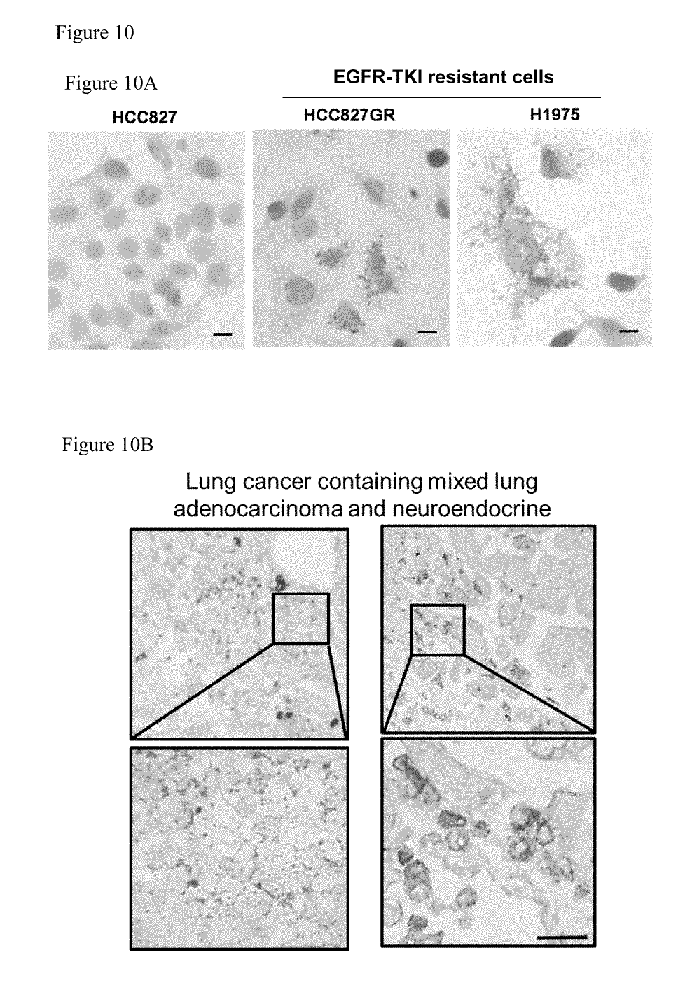

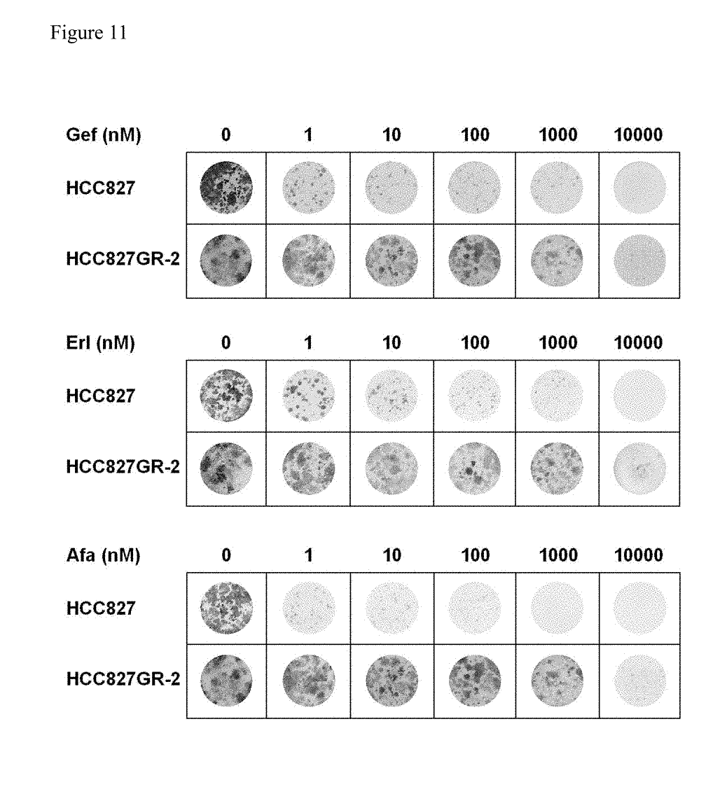

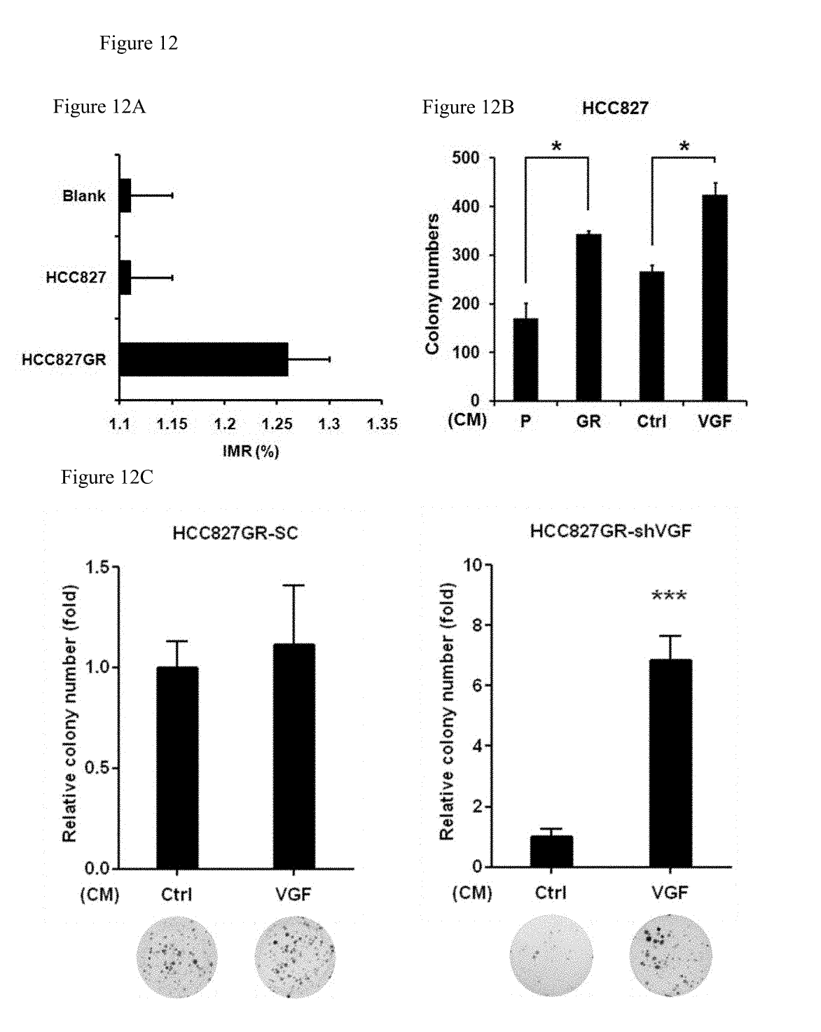

To identify genes involved in EGFR-TKI resistance and EMT in lung adenocarcinoma, a gene expression profiling assay followed by Q-PCR analysis were performed in HCC827GR versus HCC827 cells (FIG. 3A, upper). The inventors discovered that VGF, a neurosecretory protein, is highly enriched in the gefitinib resistant HCC827GR, when compared to parental HCC827 cells. Q-PCR and immunoblotting analyses showed that VGF was 10-fold differentially expressed in HCC827GR higher than in HCC827 cells (FIG. 3A). Consistently, the expression of VGF was elevated in the independently isolated TKI resistance HCC827 cells (FIGS. 3B and 10). Immunomagnetic reduction (IMR) assay displayed that HCC827GR secreted more VGF than did HCC827 in the condition medium (FIG. 12A). The inventors further examined VGF levels in cell lines derived from different subtypes of lung cancer. The inventors observed that VGF was significantly highly expressed in cell lines from SCLC compared to those adenocarcinoma and squamous cell carcinoma, while a few of adenocarcinoma cells exhibited high levels of VGF expression (FIG. 3C). To examine whether VGF levels are associated with EGFR-TKI resistant status in lung cancer cells, IC50s of gefitinib in various EGFR-mutated lung adenocarcinoma cell lines were determined (FIG. 3D, left). Q-PCR analysis revealed that VGF levels were low in TKI sensitive cells but high in resistant cells (FIG. 3D, right). These data suggest a possible association between VGF expression and EGFR-TKI resistance in lung adenocarcinoma cells. To study the role of VGF in EGFR-TKI resistance, VGF was knocked down in HCC827GR cells, followed by gefitinib-mediated cell viability analysis (FIG. 3E). Cell viability assay revealed that VGF-silencing rendered HCC827GR cells sensitive to gefitinib (FIG. 3F). These data suggest the participation of VGF in EGFR-TKI resistance.

VGF Prevents TKI-Induced Apoptosis.

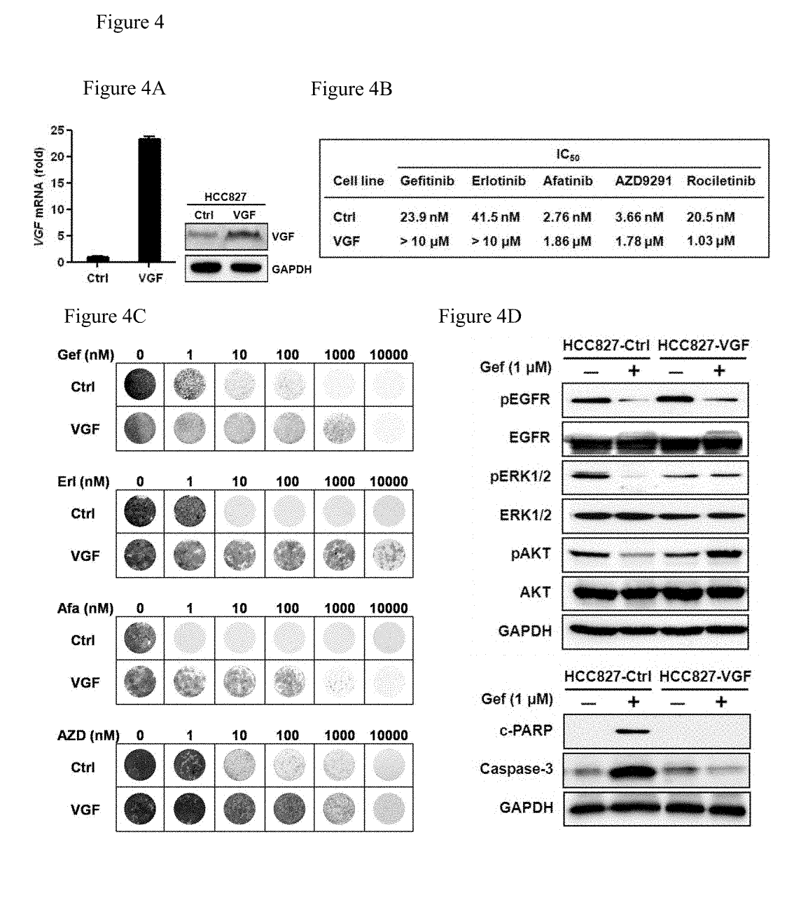

To further confirm the role of VGF in EGFR-TKI resistance, the inventors ectopically expressed VGF in HCC827 cells (FIG. 4A). Cell viability assay demonstrated that VGF expression increased IC50s of the aforementioned EGFR-TKIs in HCC827 cells (FIG. 4B). Consistently, clonogenic analysis showed that VGF expression endowed HCC827 cells with enhanced EGFR-TKI resistance, indicating that VGF expression encourages EGFR-TKI resistance in lung cancer cells (FIGS. 4C and 9). The inventors further investigated the effect of EGFR-TKI treatment on EGFR-ERK or -AKT signaling in VGF-expressing HCC827 cells Immunoblotting revealed that gefitinib treatment attenuated phosphorylation of ERK but not that of AKT in VGF-expressing cells (FIG. 4D, upper). Moreover, gefitinib treatment induced the expression of cleaved PARP and activated caspase-3 in HCC827 but not in VGF-expressing cells (FIG. 4D, lower). These data demonstrated that VGF expression sustains AKT activation and prevents cells from TKI-induced apoptotic cell death.

VGF Induces EMT and Cancer Cell Dissemination

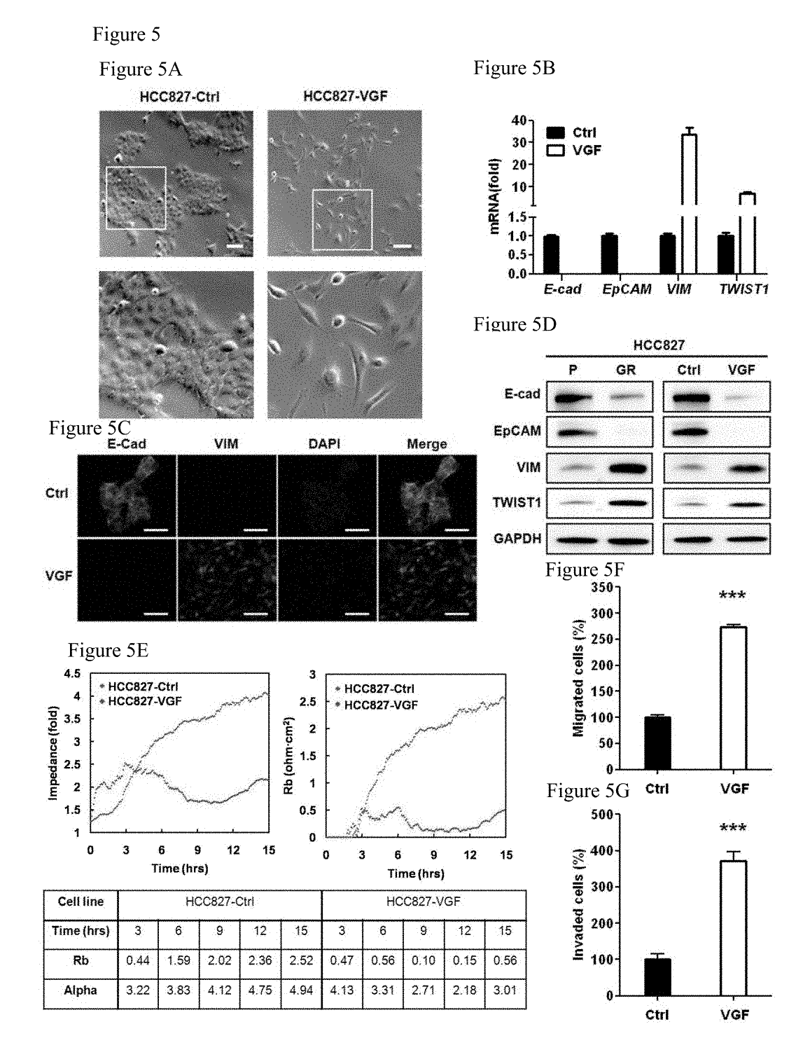

Because above data suggest that EGFR-TKI resistance can be attributed to VGF expression and associated with EMT, the inventors further characterized the effect of VGF expression on EMT and cancer cell dissemination. Phase-contrast imaging showed that the expression of VGF induced a morphological change from an epithelial phenotype to a spindle-like morphology (FIG. 5A). Q-PCR and immunoblotting assays revealed that ectopic expression of VGF attenuated the expression of E-cadherin and EpCAM, while elevating levels of Vimentin and TWIST1 (FIGS. 5B and 5D). Immunofluorescence staining showed that VGF expression induced the switching expression from E-cadherin in HCC827 cells towards Vimentin in VGF-expressing cells, supporting that VGF induces EMT (FIG. 5C). ECIS analysis displayed that VGF expression diminished impedance (FIG. 5E, upper left) and attenuated Rb level in HCC827, indicating a loss of barrier function in VGF-expressing cells (FIG. 5E, upper right and bottom). Transwell assays revealed that VGF expression encouraged migration and invasion in lung cancer cells (FIGS. 5F and 5G). Our findings indicate that VGF induces EMT and encourages cancer cell dissemination.

VGF-Silencing Attenuates Tumor Growth

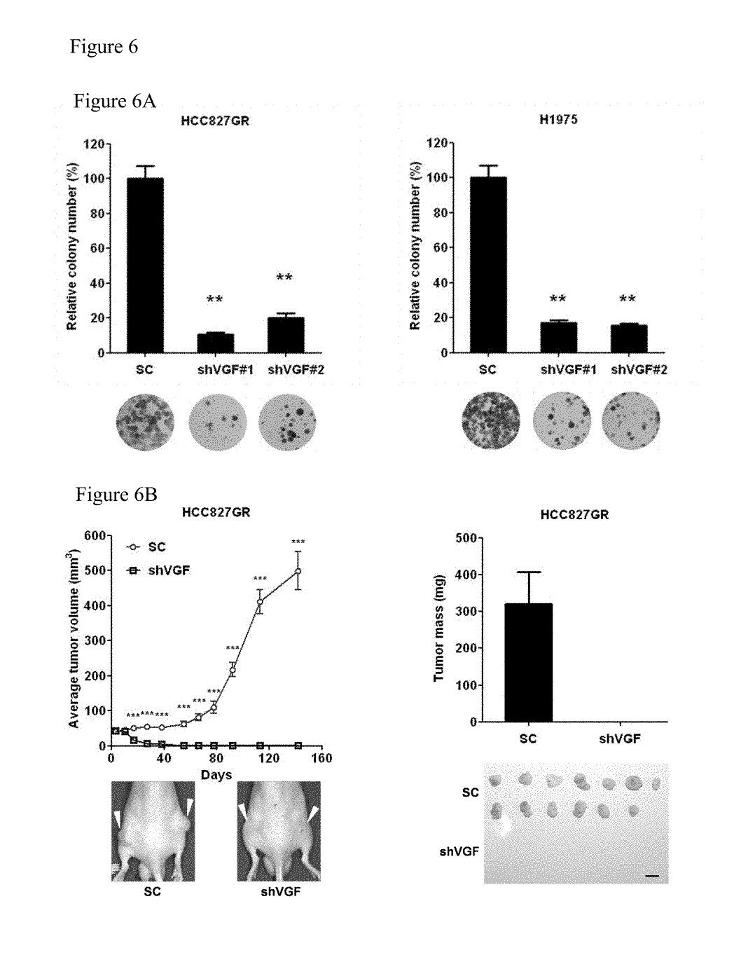

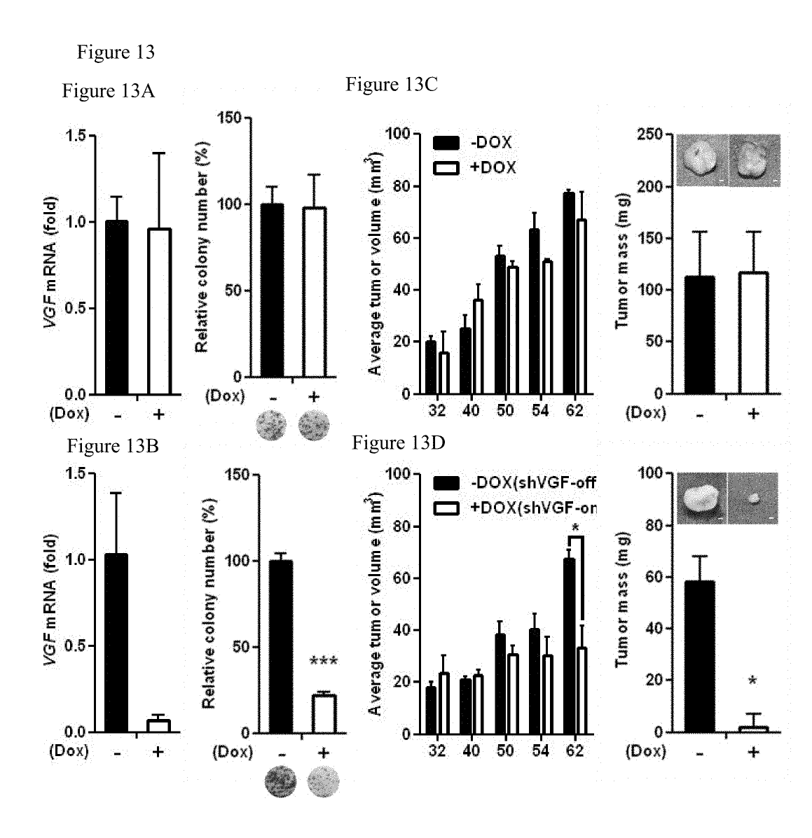

To evaluate the biological significance of endogenous VGF in EGFR-TKI resistant cells, the inventors nullified the VGF expression and tested its effect on cell growth. Clonogenic assays showed that knockdown of VGF attenuated cell growth in HCC827GR and H1975, two EGFR-TKI resistant cell lines (FIG. 6A). Treatment of VGF-silenced HCC827GR with condition medium from VGF-transfected HEK293T cells rescued cells from growth arrest, suggesting that VGF is essential for cell growth in vitro (FIG. 12C). To evaluate the importance of VGF in maintaining cell growth in vivo, VGF was knocked down in HCC827GR cells; these cells were subsequently used in a subcutaneous xenograft assay conducted in immunodeficient mice. The inventors found that whereas HCC827GR cells formed tumors in this animal model, the tumor-forming ability was inhibited in VGF-silenced cells, indicating that VGF regulates tumor cell growth in vivo (FIG. 6B). The inventors further generated HCC827GR/tet-on shVGF cells in which doxycycline (Dox) induced shVGF expression to silence VGF (FIG. 13B). Clonogenic assays showed that Dox treatment attenuated cell growth in HCC827GR/tet-on shVGF but not in control HCC827GR/tet-on cells (FIGS. 13A and 13B). To test whether VGF could function as a therapeutic target, HCC827GR/tet-on shVGF cells were subjected to a xenograft animal assay. When palpable tumor bulges were observed in the host mice, shVGF was induced in the xenograft tumors through Dox treatment. The inventors found that knockdown of endogenous VGF with Dox treatment attenuated tumor growth, causing decrease of tumor weight from HCC827GR/tet-on shVGF cells while Dox alone had no effect on tumor growth of HCC827GR/tet-on control cells (FIGS. 13C and 13D). Our findings support the notion that VGF is essential for cell growth and tumor growth in a subset of EGFR-TKI resistant lung cancer cells.

VGF Correlates with Advanced Tumor Grades and Poor Survival Outcomes in Lung Adenocarcinoma

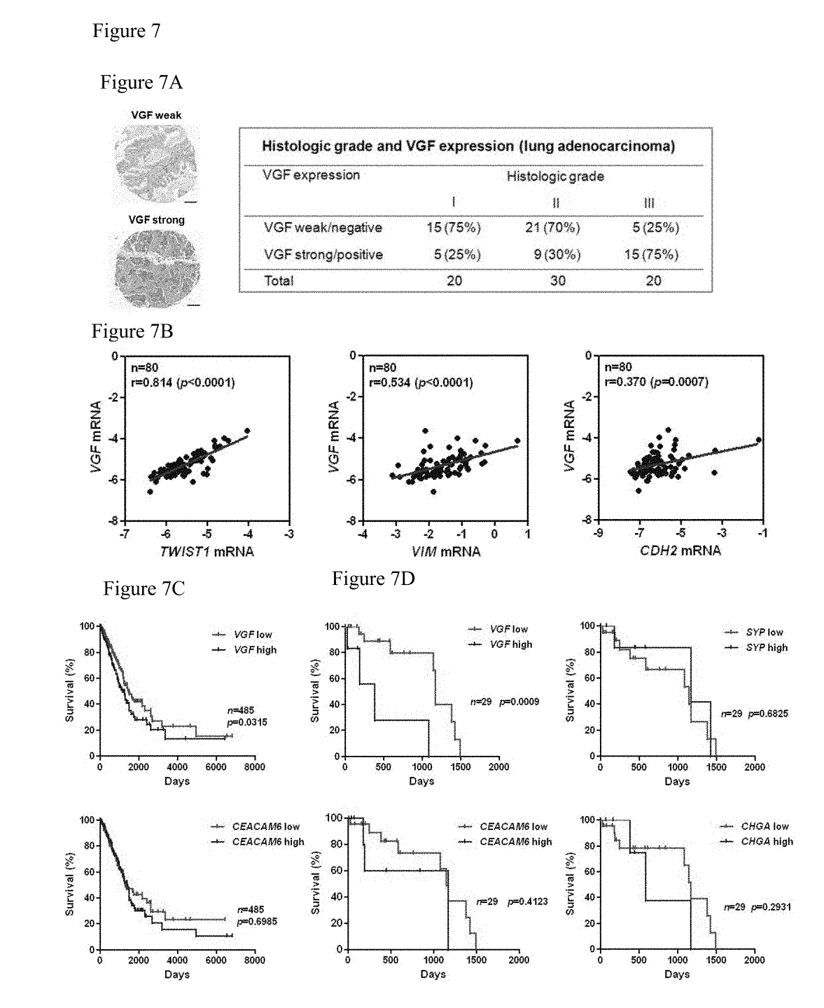

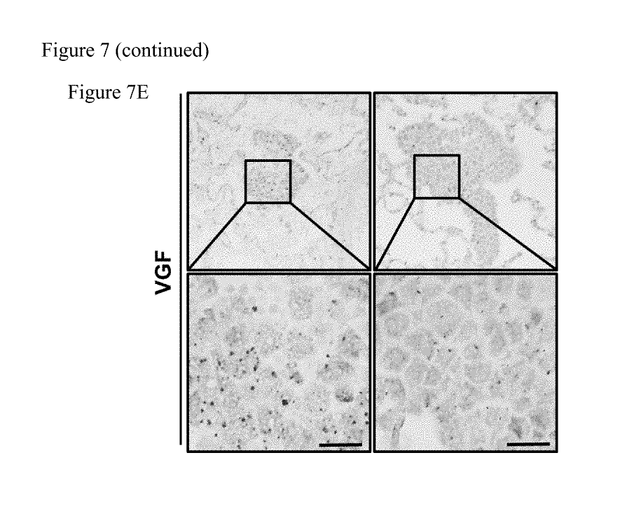

To characterize the role of VGF in lung tumor progression, the inventors measured the expression of VGF in lung adenocarcinoma by immunohistochemistry (IHC) analysis of a panel of 70 specimens. IHC staining revealed that the majority of lung adenocarcinoma with high VGF expression contained advanced tumor grades (FIG. 7A, left). Chi-square analysis indicated the association of VGF levels with pathologic grades is significant (p=0.001) (FIG. 7A, right). Moreover, RNA in situ hybridization analysis (left) and immunohistochemical staining (right) revealed that VGF was expressed in a poor differentiated lung cancer containing mixed lung adenocarcinoma and neuroendocrine carcinoma (FIG. 10B). The aforementioned data displayed that VGF induced EMT in lung cancer cells. The inventors further validated the correlation of VGF with EMT markers in primary lung adenocarcinoma.

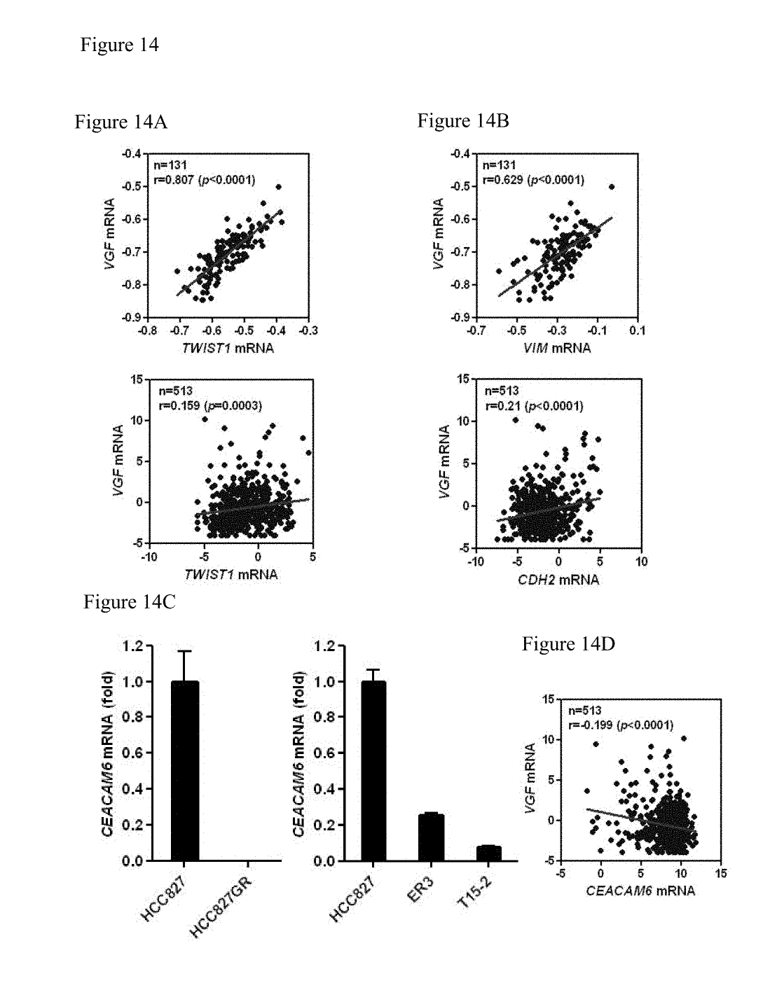

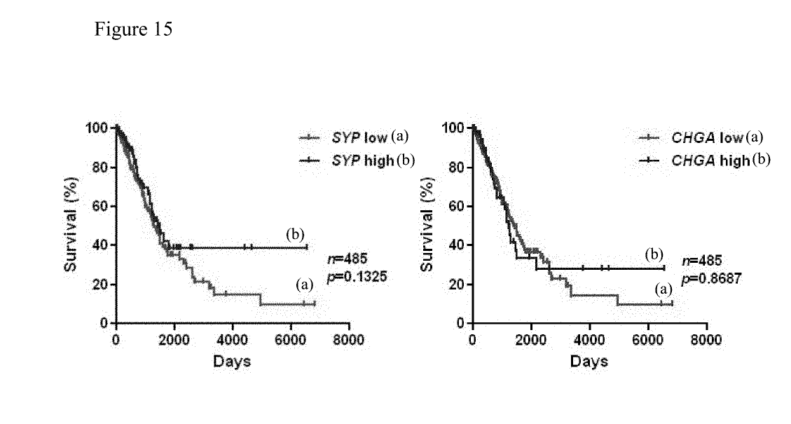

Correlation analysis showed the existence of positive correlations of VGF with TWIST1, VIM, and CDH2 (FIGS. 7B, 14A, and 14B). Because CEACAM6 is currently used as a biomarker for diagnosis and prognosis in lung adenocarinoma, the inventors further examined its expression in EGFR-TKI resistant versus sensitive cells. Q-PCR assay revealed that the expression of CEACAM6 was lost in EGFR-TKI resistant HCC827GR and independently selected cells compared to the parental HCC827 (FIG. 14C). Correlation analysis revealed that VGF was negatively associated with CEACAM6 (FIG. 13D). Kaplan-Meier survival analysis was then conducted to determine the prognostic significance of the expression of VGF versus CEACAM6 in lung adenocarcinoma. The inventors found that patients in the VGF expression correlated with poor overall survival in patients; in contrast, the expression of CEACAM6 did not predict a poor survival outcome in lung adenocarcinoma (FIG. 7C). Moreover, Kaplan-Meier survival analysis displayed that VGF expression was associated with poor survival outcome In EGFR-mutated lung adenocarcinoma, while the expression of CEACAM6 or traditional neuroendocrine markers, such as Synatophysin (SYP) and Chromogranin (CHGA), did not correlate with the survival outcome (FIG. 7 D). These data suggest a possible participation of VGF in lung cancer malignancy Immunohistochemical staining showed that VGF is expressed in EGFR-TKI resistant lung adenocarcinomas, harboring EGFR mutations (FIG. 7E).

Although EMT and neuroendocrine transformation have been linked to EGFR-TKI resistance, the mechanism is not clear. In this invention, the inventors found that VGF, a neuroendocrine protein, was highly expressed in EGFR-TKI resistant lung adenocarcinoma cells, and silencing of VGF rendered cells sensitive to EGFR-TKI treatment. Ectopic expression of VGF endowed cells with EGFR-TKI resistance and EMT. Our findings revealed for the first time that VGF functions as an emerging factor in EGFR-TKI resistance and EMT in lung adenocarcinoma.

VGF was originally identified in neuron and neuroendocrine cells, responsible for normal metabolism as well as cell survival and proliferation in the hippocampus. Moreover, VGF was reported to protect neuron cells against ER stress-Induced cell death, suggesting its involvement in stress-induced cell survival. In lung cancer, VGF was first detected in neuroendocrine lung carcinoma cell lines via proteomic analysis, while the biological and clinical significance of VGF in tumors have not been known. In this invention, the inventors found that VGF was highly expressed in EGFR-TKI resistant HCC827GR, but not in its parental HCC827. The inventors discovered that the expression of VGF activated AKT survival signaling, preventing cells from EGFR-TKI induced apoptosis in lung adenocarcinoma cells. The inventors found that VGF-containing conditioned medium can promote cell growth in the low serum culture (FIG. 12B); moreover, silencing of VGF in HCC827GR cells attenuated tumor cell growth in vitro and in vivo. These data highlight an essential role of VGF in growth and survival in HCC827GR cells. In addition, the inventors observed that H1975, an EGFR-TKI resistant lung adenocarcinoma cell line harboring both EGFR L859R and T790M mutations, contained a high level of VGF, while knockdown of VGF in H1975 cells attenuated cell growth (FIG. 6B, right). All these data indicate that VGF not only functions as a neurotrophin factor but also works as an autocrine or paracrine factor to encourage cell growth and survival in a subset of lung adenocarcinoma.

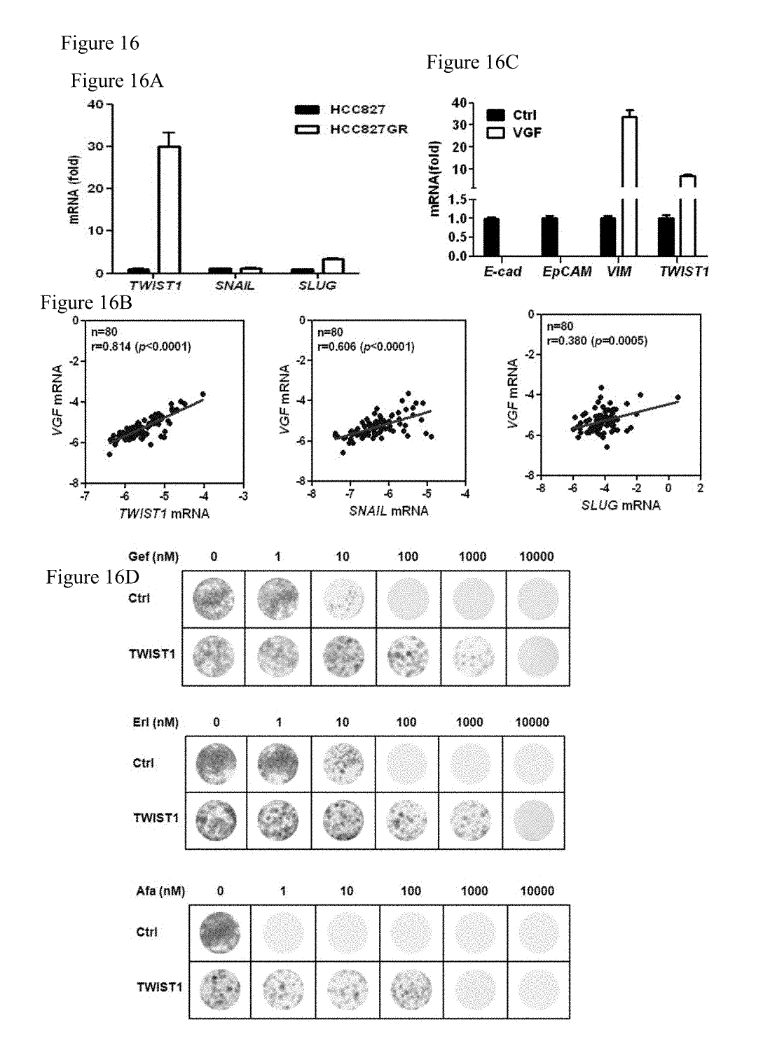

EMT has been linked to EGFR-TKI resistance; however, the mechanism is not known. Here, the inventors observed that during EGFR-TKI selection, EMT phenotypic conversion occurred in HCC827GR cells, which contain high levels of VGF and TWIST1; thus, HCC827GR cells developed EGFR-TKI resistance in a non-T790M dependent manner (FIGS. 1 and 8). The inventors found that ectopic expression of VGF in HCC827 cells not only conferred HCC827 cells resistant to EGFR-TKIs but also induced EMT phenotypic alteration accompanied with TWIST1 upregulation (FIGS. 5 and 16). TWIST1 has been reported to regulate normal cell differentiation and EMT; in addition, TWIST1 encourages cancer cell survival and dissemination. Here, the inventors observed that ectopic expression of VGF induced TWIST1 (FIG. 16), suggesting the involvement of VGF-TWIST1 signaling in cancer cell survival and dissemination. Moreover, TWIST1 expression encouraged EGFR-TKI resistance (FIG. 16C). These data indicate a potential participation of TWIST1 in VGF-mediated TKI resistance and EMT.

The human carcinoembryonic antigen (CEA), mainly refereed to CEACAM5 and CEACAM6 with shared antigenic determinants, has been wildly used as a tumor marker in cancer colorectal as well as in lung cancer while CEACAM6 expression is higher than CEACAM5 in lung adenocarcinoma. However, the use of CEA as a prognostic and predictive marker in lung cancer patients is debated. The inventors observed that CEACAM6 expression was lost in HCC827GR cells and other independently selected EGFR-TKI resistant cells compared to the parental HCC827 cells (FIG. 14C). These data suggest that CEACAM6 expression could be affected by non-T790M mediated EGFR-TKI resistance. Moreover, VGF was negatively correlated with CEACAM6 expression in the primary lung adenocarcinoma (FIG. 14D). The inventors found that the expression of CEACAM6 did not correlate with overall survival in patients while VGF expression predicted a poor survival in patients of lung adenocarcinoma, even in the EGFR-mutated subpopulation. Recently, VGF was detected in triple negative breast cancer and displayed as a better neuroendocrine biomarker than CHGA and SYP. In this invention the inventors found that VGF, but not CHGA or SYP, correlated with a poor survival in patients of lung adenocarcinoma. These data suggest that VGF could function as a predictive biomarker in lung adenocarcinoma.

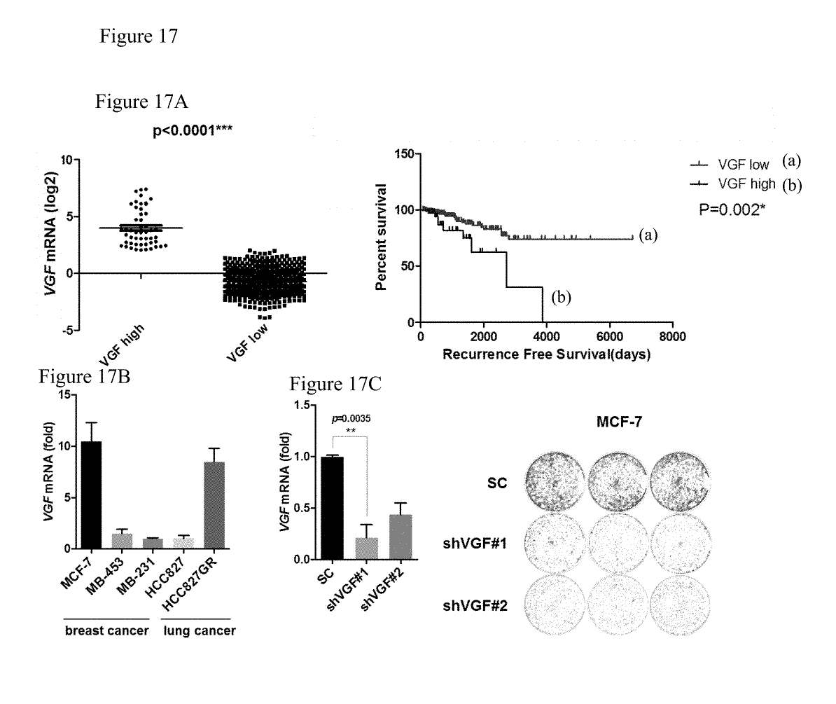

The inventors found that VGF expression predicts poor overall survival outcomes in patients with breast cancer (FIG. 17A). The inventors observed that VGF was highly expressed in not only HCC827GR lung cancer cells but also MCF7 breast cancer cells (FIG. 17B), and knockdown of VGF attenuated cell growth in MCF7 (FIG. 17C). These data suggest that VGF could function as a predictive marker and therapeutic target in breast cancer.

Taken together, the inventors found that VGF is highly expressed in a subgroup of lung adenocarcinoma cells and encourages EGFR-TKI resistance and EMT, thereby predicting a poor survival. These findings provide new insights for the role of VGF into oncogenesis of lung cancer with the potential to serve as a biomarker and therapeutic target for lung cancer intervention.

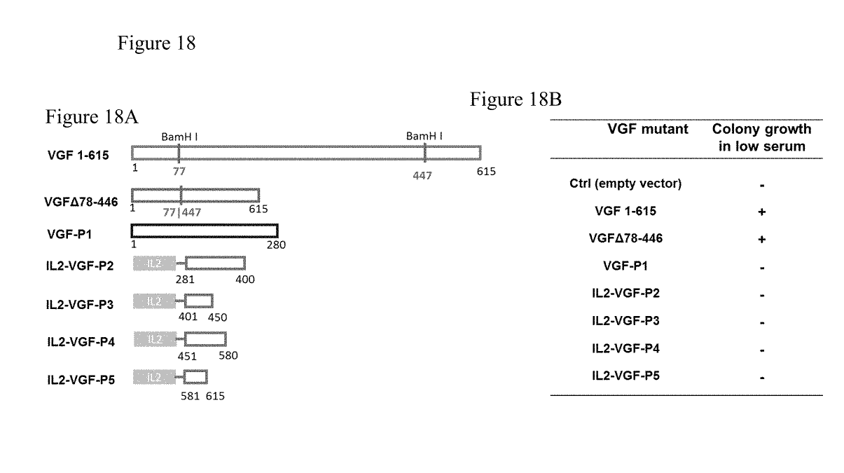

The effect of VGF mutants on low serum stress was examined (FIG. 18). Clonogenic analysis of HEK293T cells transfected with expression vector encoding empty control, full-length VGF cDNA, or truncated VGF cDNA under the growth of DMEM supplement with 1% FBS for 10 days were summarized in FIG. 18B. The inventors found that VGF 1-615 and VGFΔ78-446 mutant transfected HEK293T cells survived in the low serum culture, suggesting that VGFΔ78-446 mutant possesses the same effect as full length VGF 1-615.

What is claimed is:

1. A method of inhibiting tumor progression in a subject suffering from VGF expressing cancers, comprising administering an antagonist of VGF to the subject.

2. The method of claim 1, wherein the antagonist of VGF is antibody, small molecule compound, siRNA, shRNA or antisense RNA against VGF.

3. The method of claim 1, wherein the tumor progression comprises tumor growth, cancer dissemination, metastasis and drug resistance.

4. The method of claim 3, wherein the drug resistance comprises EGFR-TKI resistance.

5. The method of claim 1, wherein the VGF-expressing cancers comprise VGF-expressing cancers originated from lung, or breast.

6. A method of predicting or determining tumor progression state in a subject suffering from VGF expressing cancers, comprising:
(a) providing a sample from the subject; and
(b) measuring an expression level of VGF gene in the sample from the subject using reagents specific for VGF gene product that are selected from the group consisting of probes, primers, antibodies, antibody fragments and antibody coated beads, wherein the VGF gene product is VGF mRNA or VGF protein expression, wherein positive detection of VGF gene product is indicative of tumor progression.

7. The method of claim 6, wherein the expression level of VGF gene is determined by quantitative real-time PCR or in situ hybridization for VGF mRNA.

8. The method of claim 6, wherein the expression level of VGF gene is determined by immunoblotting, immunohistochemistry, or immunomagnetic reduction for VGF protein.

9. The method of claim 6, wherein the sample comprises tissue sample, serum, pleural effusion, or ascites.

10. A kit for predicting or determining tumor progression state in a subject suffering from VGF expressing cancers comprising reagent specific for VGF gene product, wherein the reagent specific for VGF gene product comprises an antibody against VGF protein, a nucleic acid probe for hybridizing to VGF mRNA, a primer pair for amplifying VGF cDNA.

11. The kit of claim 10, wherein the VGF-expressing cancers comprise VGF-expressing cancers originated from lung or breast.
